(19) United States
(12) Patent Application Publication
(10) Pub. No.: US 2013/0034208 A1
(43) Pub. Date: Feb. 7, 2013

(54) DEVICE USING X-RAYS TO HIGHLIGHT SOFT-TISSUE PARTS IN MEDICAL RADIOTHERAPY

(71) Applicants: Oliver Heid, Erlangen (DE); Jürgen Heller, Spardorf (DE)

(72) Inventors: Oliver Heid, Erlangen (DE); Jürgen Heller, Spardorf (DE)

(21) Appl. No.: 13/641,488

(22) PCT Filed: Mar. 23, 2011
(86) PCT No.: PCT/EP2011/054392

§ 371 Date: Oct. 16, 2012

(30) Foreign Application Priority Data
Apr. 16, 2010 (DE) 10 2010 015 224.2

(51) Int. Cl. A61B 6/08

(52) U.S. Cl. 378/62; 378/91

(57) ABSTRACT

A device and method for using X-rays to highlight soft-tissue parts in medical imaging are provided. The device may be implemented in radiotherapy equipment or used in radiotherapy. A control of the radiation dose needed for the therapy may involve phase-contrast imaging using X-rays to highlight soft-tissue parts. The result of the imaging by highlighting soft-tissue parts can be used for real-time and non-real-time planning of therapy and for adapting the treatment plan or the radiation dose. The radiation dose control may include anatomical imaging for locating tumours before, during and after irradiation and/or real-time adaptation of the treatment plan based on imaging that highlights soft-tissue parts. The positioning and arrangement of the combination of X-ray sources and detector in such a radiotherapy apparatus and of an accelerator may be independent of one another. The accelerator makes it possible to cover the entire body of the patient with X-rays.

### CROSS-REFERENCE TO RELATED APPLICATIONS

This application is a U.S. National Stage Application of International Application No. PCT/EP2011/054392 filed Mar. 23, 2011, which designates the United States of America, and claims priority to DE Patent Application No. 10 2010 015 224.2 filed Apr. 16, 2010. The contents of which are hereby incorporated by reference in their entirety.

### TECHNICAL FIELD

This disclosure mainly relates to a device using X-rays to highlight soft-tissue parts in medical imaging. This device and an associated method can, e.g., be used in a radiotherapy unit or utilized in radiotherapy.

### BACKGROUND

In general, within the scope of radiotherapy, a target region within the human body is to be irradiated in order to combat diseases, particularly cancer. Here, a high radiation dose is generated in a targeted fashion in an irradiation center (isocenter) of an irradiation apparatus or radiotherapy unit. A radiotherapy unit applies medically ionizing radiation to the human in order to cure diseases or to delay their advance, particularly in the case of tumors. Here, gamma radiation, X-ray radiation and electrons are predominantly used as ionizing, high-energy rays. It is also possible to use installations for treatment with neutrons, protons and heavy ions.

In order to treat a tumor, for example, a radiotherapy unit should realize a specific desired dose distribution in a target volume. The problem of the irradiation target in the body being movable often occurs during irradiation. Thus, for example, a tumor in the abdominal region is displaced during respiration. Secondly, a tumor can also have grown or already shrunken in the time between irradiation planning and actual irradiation. It is therefore possible to control the position of the irradiation target in the body during the irradiation by means of imaging in order to control the beam appropriately or to be able, where necessary, to interrupt the irradiation and thus improve the success of the therapy.

A goal in radiotherapy is a treatment guided with the aid of real-time images, without the need for repositioning the patient during the treatment. There are either radiation therapy systems with integrated X-ray imaging or separate computed tomography or magnetic resonance imaging, which support the treatment planning. However, radiation therapy systems with integrated X-ray imaging do not supply high-resolution soft-tissue contrast images for precise treatment or irradiation, and they do not satisfy a necessary option for adapting the treatment in real time on the basis of the created images. That is to say that an adaptation for respiration or patient movement during the treatment is not yet possible at this moment in time. There are radiation therapy systems with integrated conventional X-ray imaging with conventional characteristic contrast imaging, which are based on the absorption of photons, with the photoelectronic process being used for imaging the target region of interest. This use is disadvantageous to the extent that the generated contrast is unsuitable for visualizing soft-tissue parts and is limited in its precision during radiation treatment. Moreover, it proves impossible to achieve real-time adaptation of the treatment plan. An ultrasound apparatus can also be used as imaging medium for monitoring the treatment or therapy. However, these only provide a restricted solution to the problem. Ultrasound imaging lacks the penetration depth for many applications. Furthermore, various radiation therapy systems with integrated magnetic resonance imaging solutions are known from e.g. DE 10 2008 007 245 A1. The high quality of the soft-tissue highlighting in magnetic resonance imaging is useful for identifying soft-tissue parts which should be treated by radiotherapy. These approaches are very complicated and complex.

### SUMMARY

In one embodiment, a radiotherapy device comprises imaging means, based on X-ray beams, for highlighting soft-tissue parts in a target region, which are configured such that the imaging means are embodied for phase-contrast imaging.

In a further embodiment, in respect of the target region, the imaging means can be positioned independently of the device means for radiotherapy. In a further embodiment, the imaging means have at least one X-ray source and at least one detector, which have a static arrangement with respect to one another, but can together be moved freely and/or positioned in respect of the device means for radiotherapy. In a further embodiment, the device has a control apparatus or a reception apparatus for control signals for avoiding a collision between the imaging means and the device means for radiotherapy. In a further embodiment, the imaging means have at least one monochromatic X-ray source. In a further embodiment, the imaging means have at least one coherent X-ray source. In a further embodiment, the imaging means have at least one incoherent X-ray source. In a further embodiment, the imaging means have at least one energy-suppressing detector.

In another embodiment, a method is provided for controlling the position of imaging means, based on X-ray beams, for highlighting soft-tissue parts in a target region, which are provided within a radiotherapy device for phase-contrast imaging, wherein, in respect of the target region, they are positioned independently of the device means for radiotherapy.

In a further embodiment, control signals emitted by a control apparatus bring about an avoidance of a collision between the imaging means and the device means for radiotherapy.

### BRIEF DESCRIPTION OF THE DRAWINGS

FIG. 1 shows an example of a radiation therapy unit, according to an example embodiment.

### DETAILED DESCRIPTION

Some embodiments provide a method or a device in radiotherapy which enables a treatment controlled by real-time images, wherein the imaging is intended to highlight soft-tissue parts with a sufficient accuracy. Moreover, adaptation of the treatment plan or the radiation dose in real time should be made possible.

For example, Some embodiments involve controlling the radiation dose required for the therapy, which emerges from phase-contrast imaging, based on an X-ray beam, for highlighting soft-tissue parts, which may be used in a radiation therapy device. The result of the soft-tissue part highlighting imaging can be used for real-time and not real-time therapy planning and for adapting the treatment plan or the radiation dose.

Here, radiation-dose control may comprise:
a) anatomical imaging for localizing the tumor before, during and after irradiation
b) optional: real-time adaptation of the treatment plan, based on soft-tissue part highlighting imaging.

Some embodiments provide for implementing high-quality soft-tissue part highlighting imaging such that use is made of a monochromatic X-ray source. A monochromatic X-ray source generally produces protons with a tight wavelength window in order to enable phase-contrast imaging for being able to display soft-tissue parts.

An improved soft-tissue contrast can be created by virtue of using a K absorption band.

A further embodiment provides for high-resolution soft-tissue part highlighting imaging to be implemented by an energy-suppressing X-ray detector. Scattered radiation may be suppressed as a result of a narrow photon energy range. Moreover, an increased contrast can be generated by a wavelength-dependent absorption (in particular color) or by spectroscopic information.

A further embodiment provides for high-quality soft-tissue part highlighting imaging to be implemented using a coherent X-ray source, which generates photons with a constant relative phase. An X-ray beam interferometer can be used for phase-sensitive imaging.

A further embodiment provides for implementing the high-quality soft-tissue part highlighting imaging as follows. Incoherent X-ray beam sources, which generate photons with a random phase distribution, may be used together with an interferometer. In order to be able to implement phase-contrast imaging, so-called “grating” is applied here, as a result of which a regular spatial collection of essential, identical, parallel and elongated elements is produced.

Other embodiments provide a method for controlling the position of imaging means (S, D), based on X-ray beams, for highlighting soft-tissue parts in a target region, which are provided within a radiotherapy device for phase-contrast imaging, wherein, in respect of the target region, they are positioned independently of the device means (T) for radiotherapy.

A further embodiment provides for control signals emitted by a control apparatus to bring about an avoidance of a collision between the imaging means (S, D) and the device means (T) for radiotherapy.

Certain embodiments provide one or more of the following advantages:

A radiation therapy system is provided having integrated high-quality soft-tissue part highlighting imaging, like magnetic resonance imaging, in order to enable very precise radiation treatment.

The very precise radiation therapy according to the systems and methods disclosed herein may have economically similar applications as already existing radiation therapy approaches.

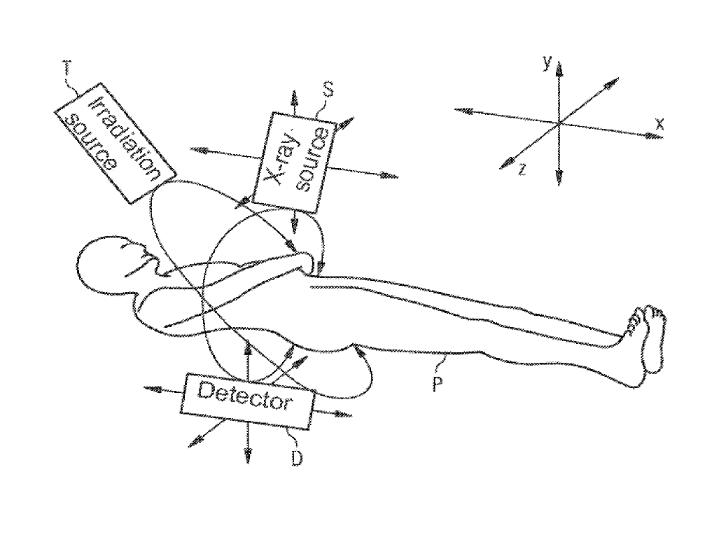

FIG. 1 shows an example of a radiation therapy unit, in which a positioning of the X-ray source S and the X-ray detector D affords the possibility of covering the whole patient body P with beams from every possible angle. This is indicated by the illustrated arrows and circles.

The illustrated accelerator or irradiation source T for the therapy renders it possible to cover the whole patient body with beams from every possible angle. This is indicated by the illustrated arrows and circles.

The positioning or arrangement of the X-ray sources and X-ray-detector combination and of the accelerator is independent of one another, wherein X-ray sources and X-ray detector can be attached statically with respect to one another (e.g. both at the “ends” of a C-arm). A hardware control or software control (not illustrated), which is integrated into the radiotherapy unit or, embodied separate from the radiotherapy unit, feeds control signals thereto, prevents a collision of the components S, D and T when these are positioned.

What is claimed is:

1. A radiotherapy device comprising: imaging means configured to highlight soft-tissue parts in a target region based, on X-ray beams,
an accelerator for radiotherapy
wherein the imaging means configured for phase-contrast imaging, and
wherein the imaging means are positioned, independently of the accelerator with respect to the target region.

2. (canceled)

3. The device of claim 1, wherein the imaging means have at least one X-ray source and at least one detector that have a static arrangement with respect to one another, and which together are freely movable and positionable relative to the accelerator.

4. The device of claim 1, wherein the device comprises a control apparatus configured to prevent a collision between the imaging means and the accelerator.

5. The device of claim 1, wherein the imaging means comprise at least one monochromatic X-ray source.

6. The device of claim 1, wherein the imaging means comprise at least one coherent X-ray source.

7. The device of claim 1, wherein the imaging means comprise at least one incoherent X-ray source.

8. The device of claim 1, wherein the imaging means at least one energy-suppressing detector.

9. A method for controlling the position of imaging means, comprising:
using imaging means to highlight soft-tissue parts in a target region based on X-ray beams,
wherein the imaging means are configured for phase-contrast imaging,
controlling an accelerator tor radiotherapy, and
controlling movement and positioning of the imaging means independently of the accelerator.

10. The method of claim 9, comprising using a control system to prevent a collision between the imaging means and the accelerator.

11. The method of claim 9, wherein the imaging means have at least one X-ray source and at least one detector that have a static arrangement with respect to one another, and which together are freely movable and positionable relative to the accelerator.

12. The method of claim 9, wherein the imaging means comprise at least one monochromatic X-ray source.

13. The method of claim 9, wherein the imaging means comprise at least one coherent X-ray source.

14. The method of claim 9, wherein the imaging means comprise at least one incoherent X-ray source.

15. The method of claim 9, wherein the imaging means comprise at least one energy-suppressing detector.
